(19) United States
(12) Patent Application Publication
(10) Pub. No.: US 2010/0022393 A1
(43) Pub. Date: Jan. 28, 2010

(54) Glyphosate applications in aquaculture

(76) Inventor: Bertrand Vick, Berkeley, CA (US)

Correspondence Address: CARR & FERRELL LLP, 2200 GENG ROAD, PALO ALTO, CA, 94303, US

(21) Appl. No.: 12/220,688

(22) Filed: Jul. 24, 2008

(51) Int. Cl. A01N 57/20; C12N 15/09; A01H 13/00; C12N 1/13; A01P 13/00

(52) U.S. Cl. 504/206; 800/278; 800/296; 435/257.2; 435/471

(57) ABSTRACT

Methods for controlling a density of algae growing in an aquatic environment are provided. Exemplary methods include applying an effective amount of glyphosate to a density of algae growing in an aquatic environment. The algae may include genus Nannochloropsis and/or Dunaliella. The algae may also include a glyphosate resistant strain of genus Nannochloropsis. The effective amount may result in an approximate concentration of between 0.1 millimolar to 0.3 millimolar glyphosate in the aquatic environment. Additionally, the aquatic environment may include seawater. The glyphosate may be applied to the aquatic environment before and/or after the aquatic environment is inoculated with algae. Alternative methods include applying an effective amount of glufosinate to a density of algae growing in an aquatic environment.

### BACKGROUND OF THE INVENTION

1. Field of the Invention

This invention relates to molecular biology, and more specifically to glyphosate applications in aquaculture.

2. Description of Related Art

Glyphosate is generally known as a foliar-applied, translocated herbicide used to control most shoreline vegetation and several emergent weeds such as spatterdock (Nupharluteum) and alligatorweed (Alternanthera philoxeroides). Glyphosate translocates from the treated foliage to underground storage organs such as rhizomes. It is generally most effective when applied during a weed's flowering or fruiting stage. If rain falls within six hours of application, the effectiveness of glyphosate is reduced. Accordingly, glyphosate would not be expected to be effective when applied in an aquatic environment. Additionally, authorities such as the Oklahoma Cooperative Extension Service (Aquatic Weed Management, Herbicides, SRAC-361 as found at http://osufacts.okstate.edu) have cited the poor response of planktonic, filamentous, and Chara/Nitella algae to glyphosate, advocating instead the use of copper and copper complexes for controlling algal growth. Consequently, the exemplary embodiments described herein involving glyphosate applications in aquaculture are novel and non-obvious in light of prior teachings.

### SUMMARY OF THE INVENTION

Methods for controlling a density of algae growing in an aquatic environment are provided. Exemplary methods include applying an effective amount of glyphosate to a density of algae growing in an aquatic environment. The algae may include genus Nannochloropsis and/or Dunaliella. The algae may also include a glyphosate resistant strain of genus Nannochloropsis. The effective amount may result in an approximate concentration of between 0.1 millimolar to 0.3 millimolar glyphosate in the aquatic environment. Additionally, the aquatic environment may include seawater. The glyphosate may be applied to the aquatic environment before and/or after the aquatic environment is inoculated with algae. An exemplary product may include a biomass generated from algal genus Nannochloropsis cultured in an aqueous environment comprising an effective amount of glyphosate. Alternative methods include applying an effective amount of glufosinate to a density of algae growing in an aquatic environment.

### BRIEF DESCRIPTION OF THE DRAWINGS

FIG. 1 shows a graph of glyphosate concentration (X-Axis) versus measured optical density (Y-Axis) for a particular exemplary Nannochloropsis culture both before and after glyphosate application;

FIG. 2 shows a graph of glyphosate concentration (X-Axis) versus measured optical density (Y-Axis) for a particular exemplary Dunaliella culture both before and after glyphosate application;

FIG. 3 shows a graph of ammonium chloride concentration (X-Axis) versus measured optical density (Y-Axis) for a particular exemplary Nannochloropsis culture;

FIG. 4 shows a graph of ammonium chloride concentration (X-Axis) versus measured optical density (Y-Axis) for a particular exemplary Dunaliella culture;

FIG. 5 shows a graph of ammonium hydroxide concentration (X-Axis) versus measured optical density (Y-Axis) for a particular exemplary Dunaliella culture;

FIG. 6 shows a graph of ammonium hydroxide concentration (X-Axis) versus measured optical density (Y-Axis) for a particular exemplary Nannochloropsis culture;

FIG. 7 shows a graph of glufosinate concentration (X-Axis) versus measured optical density (Y-Axis) for a particular exemplary Nannochloropsis culture both before and after glufosinate application; and

FIG. 8 shows a flow chart for an exemplary method of controlling algae density in an aquatic environment.

### DETAILED DESCRIPTION OF THE INVENTION

Methods for controlling a density of algae growing in an aquatic environment are provided. Such methods may include applying an effective amount of glyphosate to the density of algae. The algae may include genus Nannochloropsis and/or Dunaliella. The algae may also include a glyphosate resistant strain of genus Nannochloropsis. The effective amount may result in an approximate concentration of between 0.1 millimolar to 0.3 millimolar glyphosate in the aquatic environment. Exemplary products may be generated that include a biomass from the Nannochloropsis cultured in the aqueous environment having an effective amount of glyphosate.

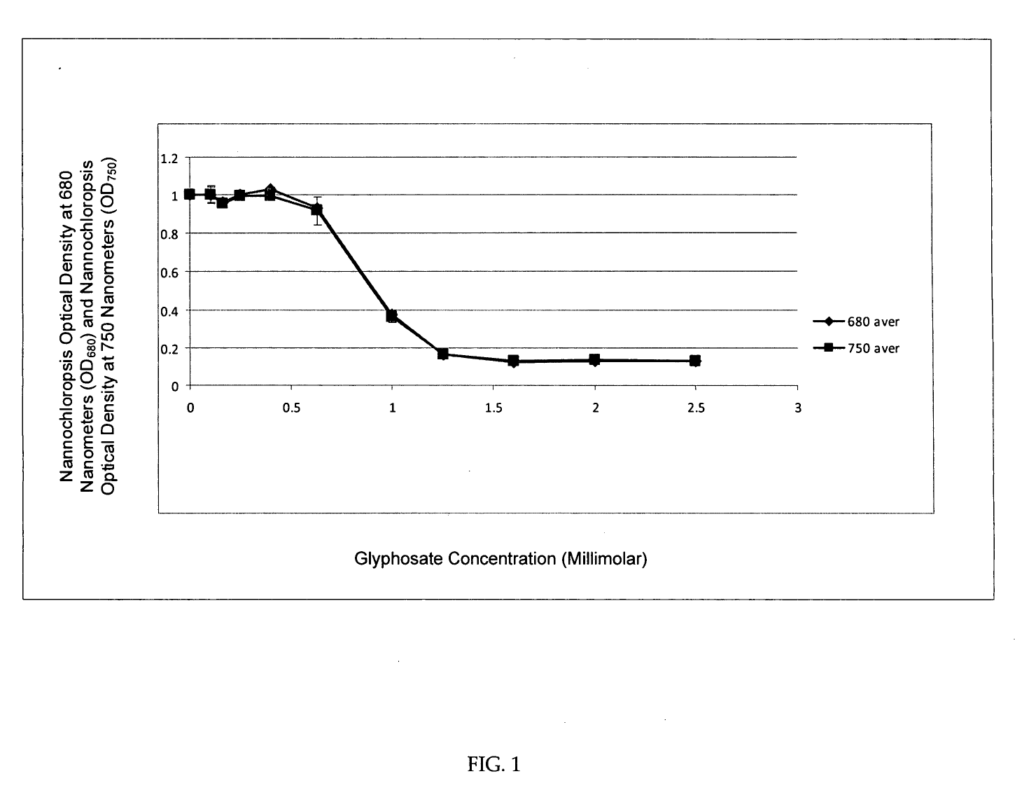

FIG. 1 shows a graph of glyphosate concentration (X-Axis) versus measured optical density (Y-Axis) for a particular exemplary Nannochloropsis culture both before and after the application of glyphosate. As shown in FIG. 1, the X-Axis shows the approximate millimolar concentration of glyphosate in an aquatic environment. The Y-Axis shows the approximate average optical density of algae growing in the aquatic environment, as measured at both 680 and 750 nanometers wavelength.

According to one exemplary method, thirty (30) microliters of a Nannochloropsis culture was introduced into seven (7) milliliters of F2 media in seawater. The mixture was distributed evenly between six well plates. Glyphosate was added at various concentrations. Additional well plates were inoculated with the same Nannochloropis culture, however, the well plates were not treated with glyphosate. After approximately six days, optical density measurements at both 680 and 750 nanometers were taken in triplicate for each of the various glyphosate concentrations. As shown in FIG. 1, glyphosate controlled (inhibited) Nannochloropsis growth. At one point on the exemplary graph shown in FIG. 1, approximately 0.8 millimolar glyphosate inhibited Nannochloropsis growth by approximately fifty percent (50%).

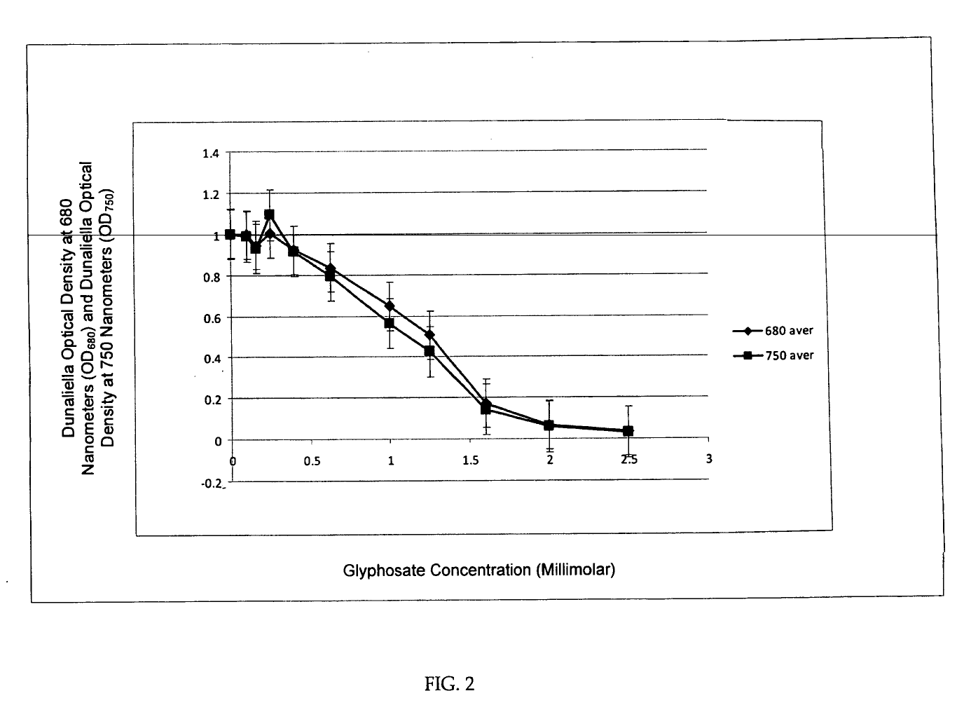

FIG. 2 shows a graph of glyphosate concentration (X-Axis) versus measured optical density (Y-Axis) for a particular exemplary Dunaliella culture both before and after the application of glyphosate. As shown in FIG. 2, the X-Axis shows the approximate millimolar concentration of glyphosate in an aquatic environment. The Y-Axis shows the approximate average optical density of algae growing within the aquatic environment, as measured at both 680 and 750 nanometers wavelength.

According to one exemplary method, thirty (30) microliters of a Dunaliella culture were inoculated into seven (7) milliliters of F2 media within seawater. The mixture was distributed evenly between six well plates. Glyphosate was added at various concentrations. Additional well plates were inoculated with the same Dunaliella culture, but were not treated with glyphosate. After approximately six days, optical density measurements at both 680 and 750 nanometers were taken in triplicate for each of the various glyphosate concentrations. As shown in FIG. 2, glyphosate inhibited Dunaliella growth. A concentration of approximately 1.2 millimolar glyphosate inhibited Dunaliella growth by approximately fifty percent (50%).

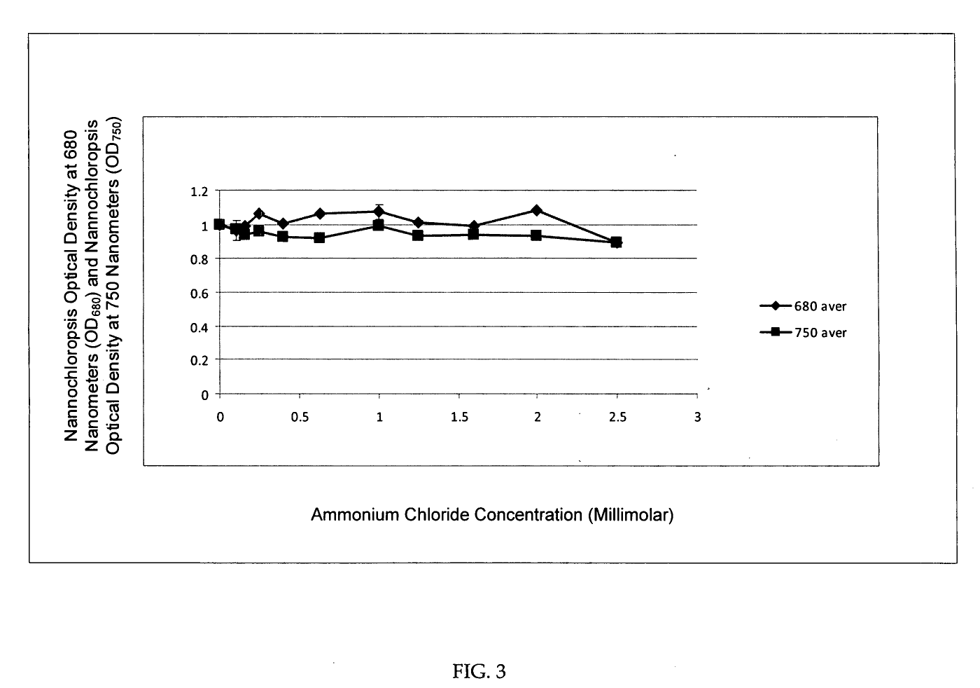

FIG. 3 shows a graph of ammonium chloride concentration (X-Axis) versus measured optical density (Y-Axis) for a particular exemplary Nannochloropsis culture. As shown in FIG. 3, the X-Axis shows the approximate millimolar concentration of ammonium chloride in an aquatic environment. The Y-Axis shows the approximate average optical density of Nannochloropsis growing in the aquatic environment, as measured at both 680 and 750 nanometers wavelength.

According to one exemplary method, thirty (30) microliters of a Nannochloropsis culture were inoculated into seven (7) milliliters of F2 media in seawater. The mixture was distributed evenly between six well plates. Ammonium chloride was added at various concentrations. Additional well plates were inoculated with the same Nannochloropis culture, but were not treated with ammonium chloride. After approximately six days, optical density measurements at both 680 and 750 nanometers were taken in triplicate for each of the various ammonium chloride concentrations. As shown in FIG. 3, ammonium chloride did not inhibit Nannochloropsis growth. Because glyphosate may be formulated in ammonium chloride, the results shown in FIG. 3 demonstrate that increased ammonium levels have little or no deleterious effect on the Nannochloropsis growth. These results strongly suggest that glyphosate is the active ingredient responsible for controlling the algal cultures described and as illustrated herein.

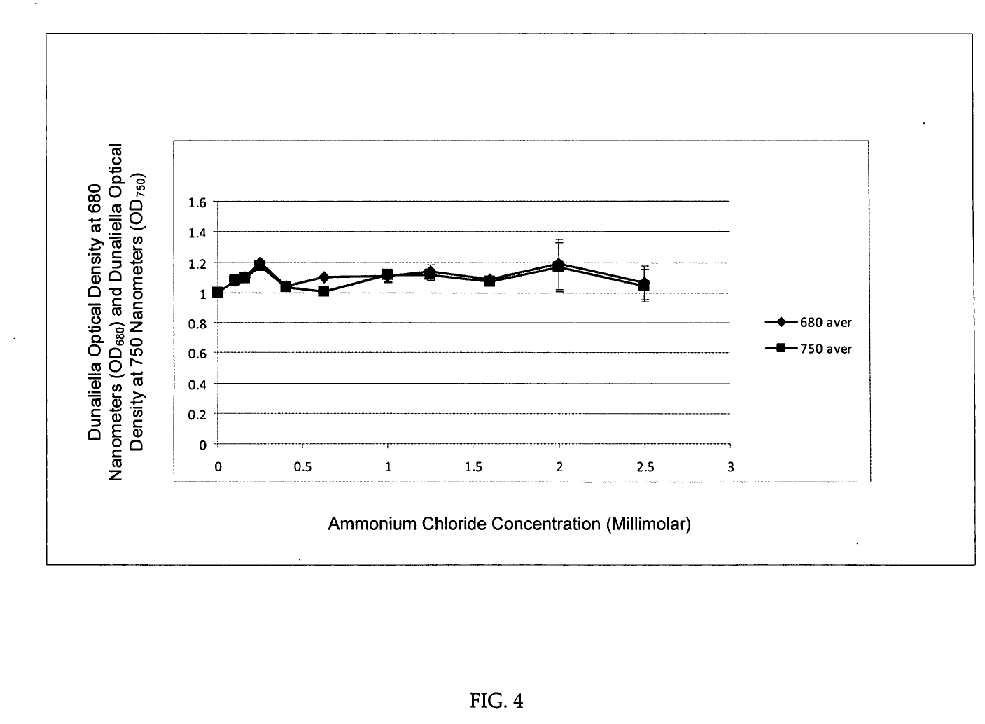

FIG. 4 shows a graph of ammonium chloride concentration (X-Axis) versus measured optical density (Y-Axis) for a particular exemplary Dunaliella culture. As shown in FIG. 4, the X-Axis shows the approximate millimolar concentration of ammonium chloride in an aquatic environment. The Y-Axis shows the approximate average optical density of Dunaliella growing in the aquatic environment, as measured at both 680 and 750 nanometers wavelength.

According to one exemplary method, thirty (30) microliters of a Dunaliella culture were inoculated into seven (7) milliliters of F2 media in seawater. The mixture was distributed evenly between six well plates. Ammonium chloride was added at various concentrations. Additional well plates were inoculated with the same Dunaliella culture, but were not treated with ammonium chloride. After approximately six days, optical density measurements at both 680 and 750 nanometers were taken in triplicate for each of the various ammonium chloride concentrations. As shown in FIG. 4, ammonium chloride did not inhibit Dunaliella growth. The results shown in FIG. 4 demonstrate that increased ammonium levels have little or no deleterious effect on the Dunaliella growth. These results strongly suggest that glyphosate is the active ingredient responsible for controlling the algal cultures described and as illustrated herein.

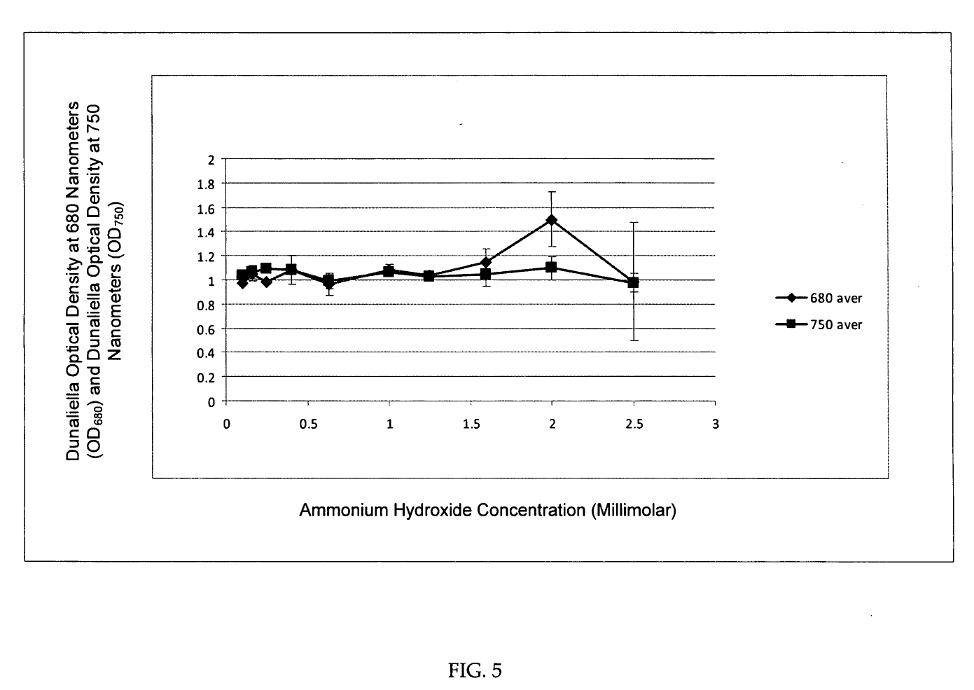

FIG. 5 shows a graph of ammonium hydroxide concentration (X-Axis) versus measured optical density (Y-Axis) for a particular exemplary Dunaliella culture. As shown in FIG. 5, the X-Axis shows the approximate millimolar concentration of ammonium hydroxide in an aquatic environment. The Y-Axis shows the approximate average optical density of Dunaliella growing in the aquatic environment, as measured at both 680 and 750 nanometers wavelength.

According to one exemplary method, thirty (30) microliters of a Dunaliella culture were inoculated into seven (7) milliliters of F2 media in seawater. The mixture was distributed evenly between six well plates. Ammonium hydroxide was added at various concentrations. Additional well plates were inoculated with the same Dunaliella culture, but were not treated with ammonium hydroxide. After approximately six days, optical density measurements at both 680 and 750 nanometers were taken in triplicate for each of the various ammonium hydroxide concentrations. As shown in FIG. 5, ammonium hydroxide did not inhibit Dunaliella growth. Because glyphosate may be formulated in ammonium hydroxide, the results shown in FIG. 5 demonstrate that increased ammonium levels have little or no deleterious effect on the Dunaliella growth. These results strongly suggest that glyphosate is the active ingredient responsible for controlling the algal cultures described and as illustrated herein.

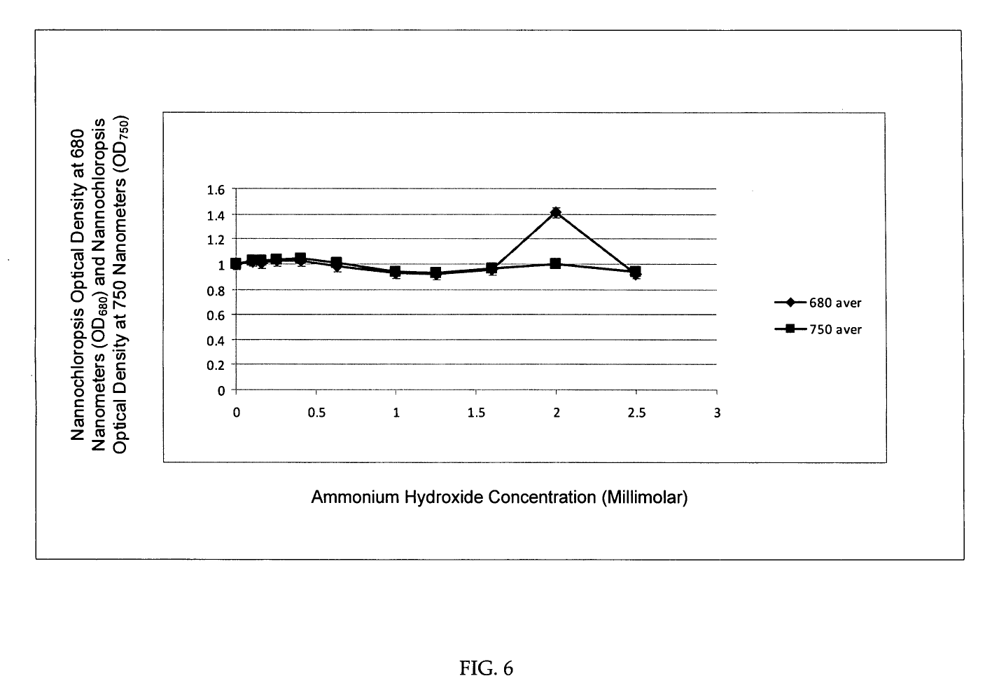

FIG. 6 shows a graph of ammonium hydroxide concentration (X-Axis) versus measured optical density (Y-Axis) for a particular exemplary Nannochloropsis culture. As shown in FIG. 6, the X-Axis shows the approximate millimolar concentration of ammonium hydroxide in an aquatic environment. The Y-Axis shows the approximate average optical density of Nannochloropsis growing in the aquatic environment, as measured at both 680 and 750 nanometers wavelength.

According to one exemplary method, thirty (30) microliters of a Nannochloropsis culture were inoculated into seven (7) milliliters of F2 media in seawater. The mixture was distributed evenly between six well plates. Ammonium hydroxide was added at various concentrations. Additional well plates were inoculated with the same Nannochloropsis culture, but were not treated with ammonium hydroxide. After approximately six days, optical density measurements at both 680 and 750 nanometers were taken in triplicate for each of the various ammonium hydroxide concentrations. As shown in FIG. 6, ammonium hydroxide did not inhibit Nannochloropsis growth. Because glyphosate may be formulated in ammonium hydroxide, the results shown in FIG. 6 demonstrate that increased ammonium levels have little or no deleterious effect on the Nannochloropsis growth. These results strongly suggest that glyphosate is the active ingredient responsible for controlling the algal cultures described and as illustrated herein.

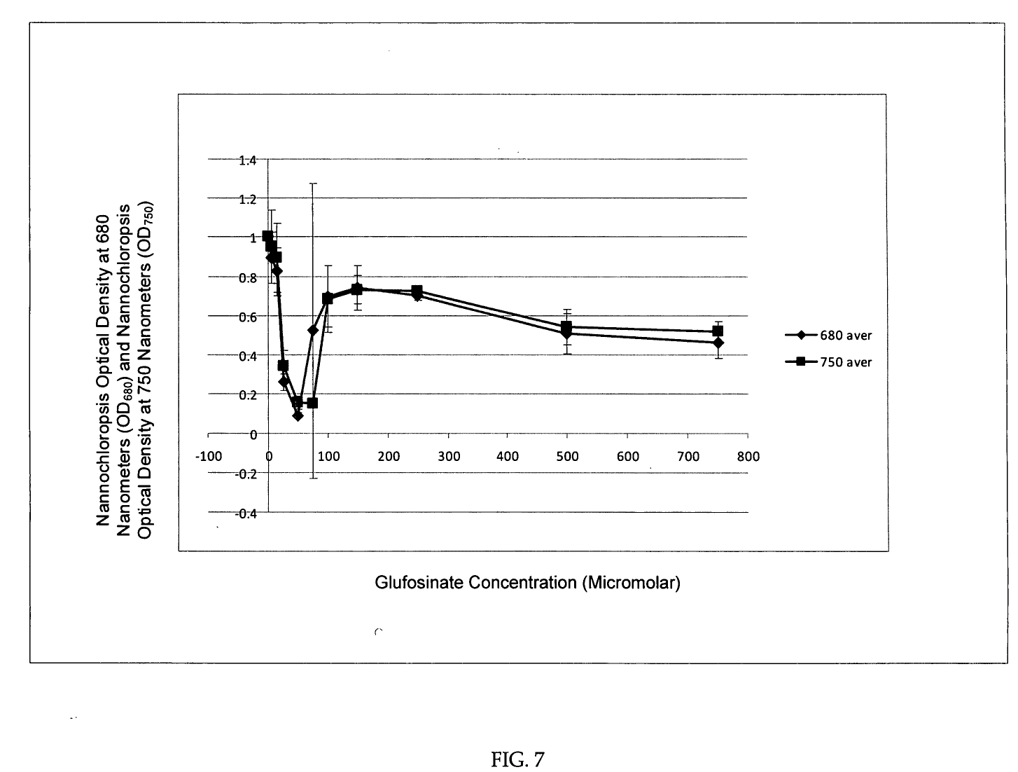

FIG. 7 shows a graph of glufosinate concentration (X-Axis) versus measured optical density (Y-Axis) for a particular exemplary Nannochloropsis culture both before and after the application of glufosinate. As shown in FIG. 7, the X-Axis shows the approximate micromolar concentration of glufosinate in an aquatic environment. The Y-Axis shows the approximate average optical density of algae growing in the aquatic environment, as measured at both 680 and 750 nanometers wavelength.

According to one exemplary method, thirty (30) microliters of a Nannochloropsis culture was introduced into seven (7) milliliters of F2 media in seawater. The mixture was distributed evenly between six well plates. Glufosinate was added at various concentrations. Additional well plates were inoculated with the same Nannochloropsis culture, however, the well plates were not treated with glufosinate. After approximately six days, optical density measurements at both 680 and 750 nanometers were taken in triplicate for each of the various glufosinate concentrations. As shown in FIG. 7, glufosinate controlled (inhibited) Nannochloropsis growth. At one point on the exemplary graph shown in FIG. 7, approximately 25 micromolar glufosinate inhibited Nannochloropsis growth by approximately fifty percent (50%).

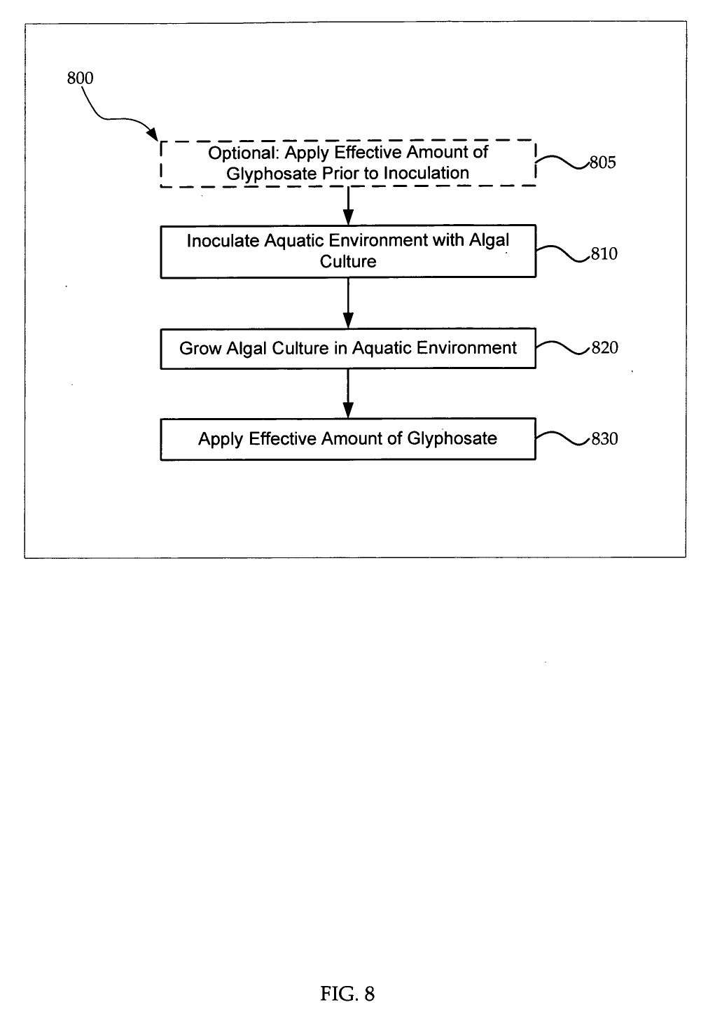

FIG. 8 shows a flow chart for an exemplary method for controlling algae density in an aquatic environment.

At optional step 805, an effective amount of glyphosate is applied to the aquatic environment before the aquatic environment is inoculated with a growing algal culture. Such-a step may be viewed as a prophylactic measure. According to one exemplary embodiment, applying an effective amount of glyphosate results in a concentration of between approximately 0.1 millimolar to 0.3 millimolar glyphosate in the aquatic environment. This step may be performed in addition to or in substitution of step 830 as described herein.

According to an alternative embodiment, an effective amount of glufosinate is applied to the aquatic environment before the aquatic environment is inoculated with a growing algal culture.

At step 810, an aquatic environment may be inoculated with an algal culture. According to various exemplary embodiments, an aquatic environment may be an open pond, a closed pond and/or a bioreactor. Further, an algal culture may comprise one or more strains of the genus Nannochloropsis, Dunaliella, and/or glyphosate-resistant strains thereof. For example, an aquatic environment may include a strain or multiple strains of algae resistant to glyphosate inhibition, such that glyphosate addition aids in maintaining a uni-algal culture. For example, a strain of algae having glyphosate resistance may survive in the presence of a particular concentration of glyphosate, while the same strain lacking glyphosate resistance may not survive in the same concentration of glyphosate. In one such case, a glyphosate resistant strain may be generated by transforming algae with a 5-endopyruvylshikimate-3 phosphate (ESPS) synthase gene which encodes a protein insensitive to glyphosate. Alternatively, a glyphosate resistant strain may be generated by mutagenesis of algal cells followed by selection with glyphosate.

According to various embodiments, outdoor algal cultures may be started with the addition of an initial, small amount of pure (virtually free from unwanted contaminant organisms) algal culture. Such an inoculum may be generated in a controlled environment, such as a laboratory or a closed system. The inoculum may be introduced into a larger volume of water that may have a predetermined salinity chosen to be optimal for the growth of the desired algal strain, and/or may be suboptimal for competing strains.

Once an algal culture is inoculated and grown to a desired density, according to some embodiments, it may either be removed (and a new culture may be started with a new inoculum), or it may be diluted according to a prescribed schedule or rate. In the first case, culturing may be performed in a batch mode and may require frequent re-inoculation. In the latter case, culturing may be performed in a continuous or semi-continuous fashion, depending on the way the dilution is actually performed. For example, assuming that the desired dilution rate is 50% daily, culture dilution may take place in one or more of several techniques. Culture dilution may take place continuously over the day (or part of the day) at a constant or at a variable rate. Culture dilution may alternatively take place semi-continuously once a day (i.e., 50% of the culture is removed and replaced with a new growth medium in a short period of time every day); semi-continuously twice a day (i.e., 25% of the culture is removed each time at two different times every day); or semi-continuously at any other desired frequency over the day.

In some embodiments, culture dilution may comprise removing the algal culture medium from the growth system—whether this is in an open pond or in a closed photobioreactor—and replacing this portion with fresh medium, which may contain all of the nutrients in the quantity sufficient for the growth of the algae between two consecutive dilutions. The nutrients may be added separately as mentioned herein. Also, by varying the salinity of the fresh medium, the salinity in the microalgal culture may be kept within a prescribed range which may be optimal for the specific algal strain and/or suboptimal for competing strains.

According to an alternative embodiment, an algal culture may comprise one or more strains of the genus Nannochloropsis, Dunaliella, and/or glufosinate-resistant strains thereof. For instance, an aquatic environment may include a strain or multiple strains of algae resistant to glufosinate inhibition, such that glufosinate addition aids in maintaining a uni-algal culture. A strain of algae having glufosinate resistance may survive in the presence of a particular concentration of glufosinate, while the same strain lacking glufosinate resistance may not survive in the same concentration of glufosinate. A glufosinate resistant strain may be generated by mutagenesis of algal cells followed by selection with glufosinate.

At step 820, the algal culture is grown in the aquatic environment. According to various embodiments, algae may be photosynthetic microorganisms that may require light (natural or artificially supplied) for growth, as well as nutrients. Other parameters such as temperature, pH, and salinity should be within acceptable ranges. The basic elements typically required for algae growth may include carbon, nitrogen, phosphorous, iron, sulfur, and/or traces of several other elements, such as magnesium, potassium, etc. Algae may reproduce asexually via mitosis, or may reproduce sexually through the formation of gametes. Generation times for asexual reproduction may range from a few hours to days.

The required nutrients may be contained in the water, supplied subsequently in dilution waters, or supplied independently of the dilution waters, in a concentration sufficient to allow the algae to grow and reach a desired final density. The amount of nutrient needed to yield a prescribed algal density may be determined by the cell quota for that nutrient. That is, by the per cent of the algal dry mass that is comprised of the element contained in the nutrient. The inverse of the cell quota is called the algae growth potential for that nutrient or element. For instance, if the desired final density is 1 gram/liter and the algal strain under consideration contains 10% nitrogen in its biomass (i.e., a cell quota of 0.1), then the initial concentration of the atomic nitrogen in the culture should be at least 0.1 gram/liter. The same calculation may be performed for all nutrients to establish their initial concentration in the culture.

Any system utilized for outdoor mass culturing of algae may be optimized for algae growth. Ambient light and temperature may not be controlled. However, the light and temperature within a culture system may depend on the actual system utilized. For example, the time averaged light intensity to which the algal culture may be exposed may be adjusted by changes in the mixing intensity and in the optical depth of the apparatus. In panel-shaped modular photobioreactors the latter may be performed by controlling the distance between two consecutive panels. On the other hand, the optical depth in open ponds may simply be the depth of the pond. Similarly, temperature in closed photobioreactors may be precisely controlled by means of indirect heat exchange while in open ponds, temperature control may be limited and may be performed by adjusting culture depth.

According to various embodiments, the salinity in the initial medium may range between 1 and 60 parts per thousand (ppt). However, to keep Nannochloropsis dominant in the culture, a salinity of 15 to 35 ppt may chosen. This may be achieved, for instance, by mixing ⅔ of seawater having a salinity of 35 ppt with ⅓ of fresh water to obtain a salinity of 23-24 ppt. Other ratios of seawater and fresh water may be used to achieve the desired level of salinity in the growth culture. The growth medium with the desired salinity may be obtained by other means, such as by adding salt to fresh water in the required amount.

After 2 to 10 days, Nannochloropsis cultures may reach a productive operating density depending on light intensity (insulation if open ponds are utilized), temperature, and the starting inoculum size. If semi-continuous or continuous culturing is utilized, the Nannochloropsis culture may be regularly diluted at a daily dilution rate ranging between 20% and 70%. Thus, a portion of the culture ranging between 20% and 70% of the entire volume may be replaced with new water that may have the same nutrient concentration of the initial medium utilized for inoculation, or the nutrient may be added separately. The salinity of the new medium may be adjusted by controlling the ratio of seawater and fresh water (or by adding the required amount of salt to fresh water or by other similar methods) to keep the salinity of the culture after the dilution in the 15-35 ppt range. For example, if the salinity of the culture before dilution has increased to 30 ppt because of evaporation and the desired dilution rate is 50%, then the new medium may need to have a salinity of about 20 ppt to achieve a salinity of 25 ppt after the dilution. This may be accomplished manually or by automatic control systems.

At step 830, an effective amount of glyphosate is applied to the growing algal culture in the aquatic environment. According to one exemplary embodiment, applying an effective amount of glyphosate results in a concentration of between approximately 0.1 millimolar to 0.3 millimolar glyphosate in the aquatic environment. According to some embodiments, if Nannochloropsis is cultured at a salinity higher than 25 ppt, the outdoor culture is more likely to be invaded by other microorganisms that will eventually outcompete Nannochloropsis. However, Nannochloropsis dominance may be maintained by applying an effective amount of glyphosate. At lower algae concentrations, less glyphosate will be required; at higher algae concentrations, more glyphosate may likely be required.

According to an alternative embodiment, an effective amount of glufosinate is applied to the growing algal culture in the aquatic environment.

While various embodiments are described herein, it should be understood that they are presented by way of example only, and not limitation. Thus, the breadth and scope of a preferred embodiment should not be limited by any of the described exemplary embodiments.

What is claimed is:

1. A method for controlling a density of algae growing in an aquatic environment, the method comprising:
applying an effective amount of glyphosate to the density of algae growing in the aquatic environment.

2. The method of claim 1, wherein the algae includes genus Nannochloropsis.

3. The method of claim 1, wherein the algae includes genus Dunaliella.

4. The method of claim 1, wherein the algae includes a glyphosate resistant strain of genus Nannochloropsis.

5. The method of claim 1, wherein applying the effective amount results in an approximate concentration of between 0.1 millimolar to 0.3 millimolar glyphosate in the aquatic environment.

6. The method of claim 1, wherein the density of the algae prior to applying the effective amount has an approximate normalized optical density of 1.0 as measured at an approximate wavelength of 750 nanometers.

7. The method of claim 1, wherein the aquatic environment includes seawater.

8. The method of claim 1, wherein the aquatic environment includes freshwater.

9. The method of claim 1, wherein the aquatic environment includes a mixture of seawater and freshwater.

10. The method of claim 1, wherein the effective amount of glyphosate in the aquatic environment is approximately 0.8 millimolar.

11. The method of claim 10, wherein the effective amount of glyphosate inhibits Nannochloropsis growth by approximately fifty percent.

12. The method of claim 1, wherein the effective amount of glyphosate in the aquatic environment is approximately 1.2 millimolar.

13. The method of claim 12, wherein the effective amount of glyphosate inhibits Dunaliella growth by approximately fifty percent.

14. The method of claim 1, wherein the aquatic environment is in a bioreactor.

15. The method of claim 1, wherein the aquatic environment is in an open pond.

16. The method of claim 1, wherein the aquatic environment is in an open vessel.

17. The method of claim 1, wherein the aquatic environment is in a closed vessel.

18. The method of claim 1, the method further comprising:
allowing the density of the algae to return to an optical density observed prior to performing the method of claim 1.

19. The method of claim 1, the method further comprising:
generating a glyphosate resistant strain of Nannochloropsis by introducing a glyphosate-insensitive 5-endopyruvylshikimate-3 phosphate (ESPS) synthase gene into wild-type Nannochloropsis.

20. A product comprising:
a biomass generated from algal genus Nannochloropsis cultured in an aqueous environment comprising an effective amount of glyphosate.

21. A method for controlling a density of algae growing in an aquatic environment, the method comprising:
applying an effective amount of glufosinate to the density of algae growing in the aquatic environment.
